(19) United States
(12) Patent Application Publication
(10) Pub. No.: US 2024/0279163 A1
(43) Pub. Date: Aug. 22, 2024

(54) ACRYLIC COMPOUND CONTAINING PERFLUOROPOLYETHER GROUP AND COMPOSITION COMPRISING THE SAME

(71) Applicant: DONGWOO FINE-CHEM CO., LTD., Iksan-si (KR)

(72) Inventors: Siae KIM, Iksan-si (KR); Min Kyu Park, Iksan-si (KR); Kyoungmoon Jung, Iksan-si (KR); Yoonseok Choi, Iksan-si (KR)

(73) Assignee: DONGWOO FINE-CHEM CO., LTD., Iksan-si (KR)

(21) Appl. No.: 18/425,912

(22) Filed: Jan. 29, 2024

(30) Foreign Application Priority Data
Feb. 6, 2023 (KR) 10-2023-0015839

(51) Int. Cl. C07C 267/00; C08K 5/29; C09D 7/63

(52) U.S. Cl. CPC C07C 267/00; C08K 5/29; C09D 7/63

(57) ABSTRACT

A compound has a central structural unit of two arylene skeletons linked by a carbodiimide group wherein a perfluoropolyether group is bonded to one end and a multifunctional (meth)acryloyl group is bonded to the other end via urethane bonds. The compound can impart good initial contact angle and wear resistance while being soluble in commonly used solvents. Thus, the compound may be useful in a coating agent as an additive for securing initial contact angle and wear resistance.

### CROSS REFERENCE TO RELATED APPLICATIONS

This application claims priority based on Korean Patent Application No. 10-2023-0015839, filed Feb. 6, 2023, the entire content of which is incorporated herein by reference in its entirety.

### TECHNICAL FIELD

The present invention relates to an acrylic compound containing a perfluoropolyether group and a composition comprising the same, and more specifically to an acrylic compound containing a perfluoropolyether group soluble in commonly used solvents and imparting excellent initial contact angle and wear resistance and a composition comprising the same.

### BACKGROUND ART

A variety of transparent coatings have been utilized for surface protection purposes on image displays such as liquid crystal displays, electroluminescent (EL) displays, plasma displays (PDs), and field emission displays (FEDs).

For example, hard coating films formed using hard coating compositions are used to protect image displays.

Since these hard coating films are often placed at the outermost edge of the image displays, mechanical properties such as wear resistance and scratch resistance, and chemical properties such as chemical resistance, as well as stain resistance against markings by fingerprints, markers, etc. and/or antifouling properties related to ease of removal are required as key performances.

Fluorine-containing polymers are known to be used for such mechanical/chemical properties, stain resistance and/or antifouling properties. However, known fluorine-containing polymers suffer from poor solubility in commonly used solvents.

Korean Patent Publication No. 10-2005-0010064 relates to an article with a composite hard coating layer and a method of forming the composite hard coating layer. Specifically, Korean Patent Publication No. 10-2005-0010064 discloses an article with a composite hard coating layer comprising a hard coating layer installed on a surface of the article and an antifouling surface layer installed on a surface of the hard coating layer, wherein the hard coating layer is a cured product of a hard coating composition comprising an active energy ray curable compound, the antifouling surface layer is a cured product of a surface material comprising a fluorine-containing polyfunctional (meth)acrylate compound and a fluorine-containing monofunctional (meth)acrylate compound, and the antifouling surface layer is adhered to the hard coating layer.

However, there is still a need to develop materials that are soluble in commonly used solvents while imparting good initial contact angle and wear resistance.

### DISCLOSURE

### Technical Problem

An object of the present invention is to provide an acrylic compound containing a perfluoropolyether group soluble in commonly used solvents and imparting excellent initial contact angle and wear resistance.

Another object of the present invention is to provide a composition comprising the acrylic compound containing a perfluoropolyether group.

Yet another object of the present invention is to provide a hard coating film and a coating film formed using the composition.

Still another object of the present invention is to provide an image display device comprising the hard coating film and/or the coating film.

### Technical Solution

According to one aspect, the present invention provides a compound represented by the following formula (I).

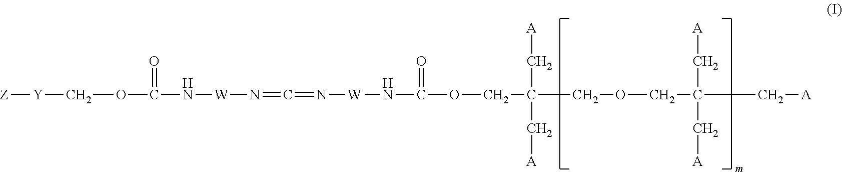

wherein,
Z is C₁-C₁₆ alkyl unsubstituted or substituted with fluorine;
Y represents —(OC₄F₈)ₐ—(OC₃F₆)_{b}—(OC₂F₄)_{c}—(OCF₂)_{d}—, wherein a, b, c, and d are each independently an integer from 0 to 200, and the sum of a, b, c, and d is 5 or more;
W represents

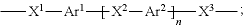

Ar¹ and Ar² are each independently arylene unsubstituted or substituted with C₁-C₄ alkyl, C₁-C₄ alkoxy, hydroxyl, or halogen;
X¹ to X³ are each independently absent, or C₁-C₄ alkylene unsubstituted or substituted with oxygen, sulfur, sulfonyl, or C₁-C₄ haloalkyl;
A is (meth)acryloyloxy; and
n and m are each independently 0 or 1.

In one embodiment of the present invention, Ar¹ and Ar² may each independently be phenylene unsubstituted or substituted with C₁-C₄ alkyl.

In one embodiment of the present invention, X¹ to X³ may each independently be absent, or C₁-C₄ alkylene unsubstituted or substituted with C₁-C₄ haloalkyl.

The compound according to one embodiment of the present invention may be a compound represented by any one of the following formulas (I-1) to (I-4).

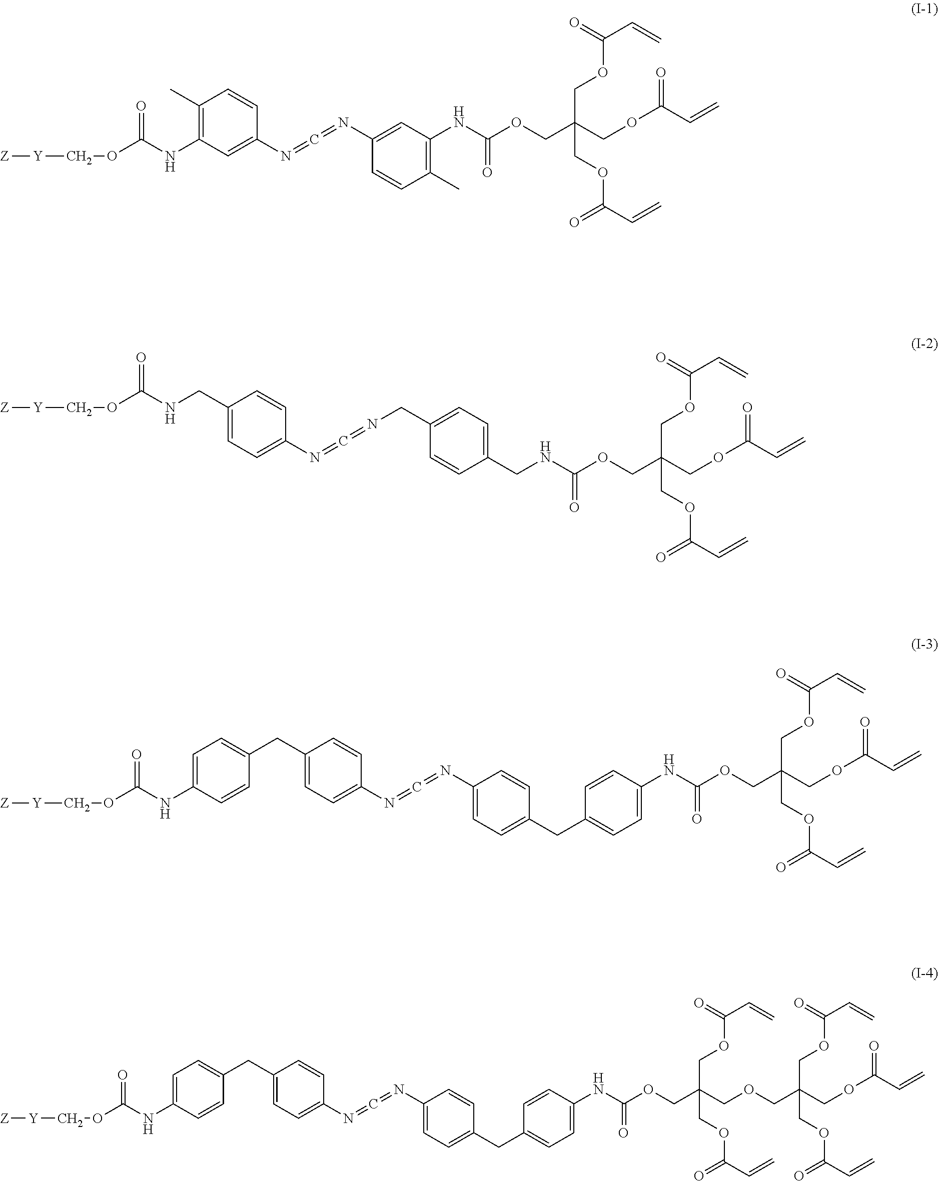

wherein,
Z is C₁-C₁₆ alkyl unsubstituted or substituted with fluorine; and
Y represents —(OC₄F₈)ₐ—(OC₃F₆)_{b}—(OC₂F₄)_{c}—(OCF₂)_{d}—, wherein a, b, c, and d are each independently an integer from 0 to 200, and the sum of a, b, c, and d is 5 or more.

According to another aspect, the present invention provides a hard coating composition comprising the compound.

In one embodiment of the present invention, the compound may be included in an amount of 2.5 to 10 wt % based on 100 wt % of the total hard coating composition.

The hard coating composition according to one embodiment of the present invention may further comprise a light-transmitting resin, a photoinitiator, and a solvent.

According to yet another aspect, the present invention provides a hard coating film formed using the hard coating composition.

According to still another aspect, the present invention provides an image display device having the hard coating film.

According to yet another aspect, the present invention provides a window of a flexible display having the hard coating film.

According to still another aspect, the present invention provides a polarizing plate having the hard coating film.

According to yet another aspect, the present invention provides a touch sensor having the hard coating film.

According to another aspect, the present invention provides a composition for forming a low reflective layer comprising the compound.

In one embodiment of the present invention, the compound may be included in an amount of 2.5 to 10 wt % based on 100 wt % of the total composition for forming a low reflective layer.

The composition for forming a low reflective layer according to one embodiment of the present invention may further comprise a light-transmitting resin, particulates having voids, a photoinitiator and a solvent.

According to yet another aspect, the present invention provides a coating film formed using the composition for forming a low reflective layer.

According to still another aspect, the present invention provides an image display device comprising the coating film.

### Advantageous Effects

The compound according to the present invention has a central structural unit of two arylene skeletons linked by a carbodiimide group with a perfluoropolyether group bonded to one end and a multifunctional (meth)acryloyl group bonded to the other end via urethane bonds, which can impart good initial contact angle and wear resistance while being soluble in commonly used solvents. In particular, the compound has good compatibility with (meth)acrylic-based clear coating agents. Therefore, the compound may be useful in a (meth)acrylic-based clear coating agent as an additive to provide initial contact angle and wear resistance.

### BEST MODE

Hereinafter, the present invention will be described in more detail.

One embodiment of the present invention relates to a compound represented by the following formula (I).

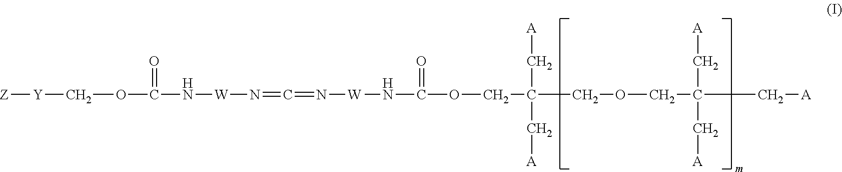

wherein,
Z is C₁-C₁₆ alkyl unsubstituted or substituted with fluorine;
Y represents —(OC₄F₈)ₐ—(OC₃F₆)_{b}—(OC₂F₄)_{c}—(OCF₂)_{d}—, wherein a, b, c, and d are each independently an integer from 0 to 200, and the sum of a, b, c, and d is 5 or more;
W represents

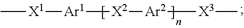

Ar¹ and Ar² are each independently arylene unsubstituted or substituted with C₁-C₄ alkyl, C₁-C₄ alkoxy, hydroxyl, or halogen;
X¹ to X³ are each independently absent, or C₁-C₄ alkylene unsubstituted or substituted with oxygen, sulfur, sulfonyl, or C₁-C₄ haloalkyl;
A is (meth)acryloyloxy; and
n and m are each independently 0 or 1.

The term “C₁-C₁₆ alkyl unsubstituted or substituted with fluorine” as used herein means a straight or branched monovalent hydrocarbon having 1 to 16 carbon atoms unsubstituted or substituted with a fluorine atom, and examples thereof include, but are not limited to, methyl, ethyl, n-propyl, i-propyl, n-butyl, i-butyl, t-butyl, pentyl, hexyl, 2-ethylhexyl, heptyl, 2-ethylheptyl, octyl, nonyl, decyl, undecyl, dodecyl, tridecyl, tetradecyl, pentadecyl, hexadecyl, trifluoromethyl, trifluoroethyl, trifluoropropyl, trifluorobutyl, and the like.

The term “C₁-C₄ alkyl” as used herein means a straight or branched monovalent hydrocarbon having 1 to 4 carbon atoms, and examples thereof include, but are not limited to, methyl, ethyl, n-propyl, i-propyl, n-butyl, i-butyl, t-butyl, and the like.

The term “C₁-C₄ alkoxy” as used herein means a straight or branched alkoxy group having 1 to 4 carbon atoms, and examples thereof include, but are not limited to, methoxy, ethoxy, n-propanoxy, and the like.

The term “arylene” as used herein includes all of divalent aromatic group, heteroaromatic group, and partially reduced derivatives thereof. The aromatic group means a 5 to 15-membered simple or fused ring, and the heteroaromatic group means an aromatic group containing at least one of oxygen, sulfur, or nitrogen. Examples of the arylene include, but are not limited to, phenylene, naphthylene, pyridinylene, furanylene, thiophenylene, indolylene, quinolinylene, imidazolinylene, oxazolylene, thiazolylene, tetrahydronaphthylene, and the like.

The term “sulfonyl” as used herein refers to a group of —S(═O)₂—.

The term “C₁-C₄ haloalkyl” as used herein means a straight or branched hydrocarbon having 1 to 4 carbon atoms substituted with one or more halogens selected from the group consisting of fluorine, chlorine, bromine and iodine, and examples thereof include, but are not limited to, trifluoromethyl, trichloromethyl, trifluoroethyl, and the like.

The term “C₁-C₄ alkylene” as used herein means a straight or branched divalent hydrocarbon having 1 to 4 carbon atoms, and examples thereof include, but are not limited to, methylene, ethylene, propylene, butylene, and the like.

In one embodiment of the present invention, Z may be C₁-C₁₆ perfluoroalkyl, in particular C₁-C₃ perfluoroalkyl.

In one embodiment of the present invention, Y is a functional group represented by —(OC₄F₈)ₐ—(OC₃F₆)_{b}—(OC₂F₄)_{c}—(OCF₂)_{d}—, a perfluoropolyether group. Here, a, b, c, and d are each independently an integer from 0 to 200, and the sum of a, b, c, and d is 5 or more, and more preferably 10 or more, such as 10 or more and 200 or less. The order of each of the above repeating units, —(OC₄F₈)—, —(OC₃F₆)—, —(OC₂F₄)— and —(OCF₂)—, may be random.

Of these repeating units, —(OC₄F₈)— may be any one of —(OCF₂CF₂CF₂CF₂)—, —(OCF(CF₃)CF₂CF₂)—, —(OCF₂CF(CF₃)CF₂)—, —(OCF₂CF₂CF(CF₃))—, —(OC(CF₃)₂CF₂)—, —(OCF₂C(CF₃)₂)—, —(OCF(CF₃)CF(CF₃))—, —(OCF(C₂F₅)CF₂)—, and —(OCF₂CF(C₂F₅))—, and preferably —(OCF₂CF₂CF₂CF₂)—. Further, —(OC₃F₆)— may be any one of —(OCF₂CF₂CF₂)—, —(OCF(CF₃)CF₂)—, and —(OCF₂CF(CF₃))—, and preferably —(OCF₂CF₂CF₂)—. Furthermore, —(OC₂F₄)— may be any one of —(OCF₂CF₂)—, and —(OCF(CF₃))—, and preferably —(OCF₂CF₂)—.

In one embodiment of the present invention, Ar¹ and Ar² may be each independently phenylene unsubstituted or substituted with C₁-C₄ alkyl, in terms of solubility in solvents, initial contact angle, and/or wear resistance.

In one embodiment of the present invention, X¹ to X³ may each independently be absent, or C₁-C₄ alkylene unsubstituted or substituted with C₁-C₄ haloalkyl, in terms of solubility in solvents, initial contact angle, and/or wear resistance.

In particular, the compound according to one embodiment of the present invention may be a compound represented by any one of the following formulas (I-1) to (I-4).

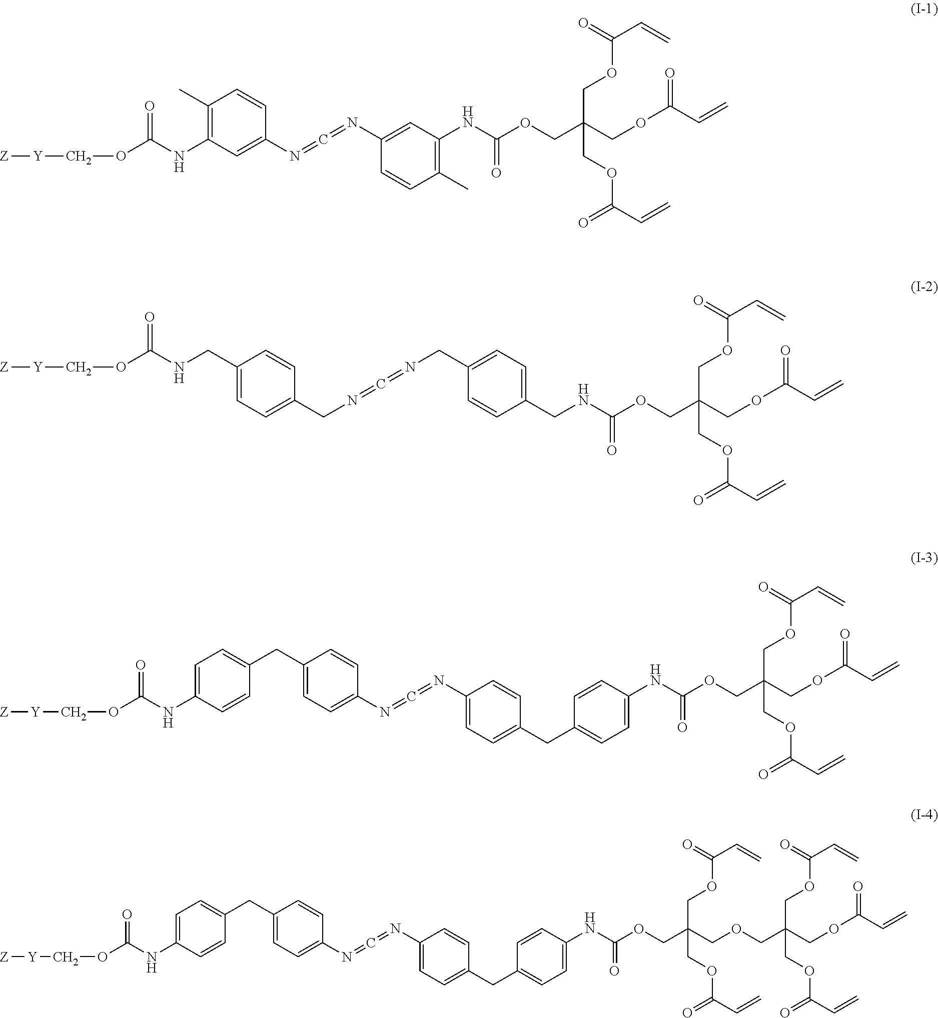

wherein,
Z is C₁-C₁₆ alkyl unsubstituted or substituted with fluorine; and
Y represents —(OC F₈)—(OC₃F₆)_{b}—(OC₂F₄)—(OCF₂)_{d}—, wherein a, b, c, and d are each independently an integer from 0 to 200, and the sum of a, b, c, and d is 5 or more.

The compound represented by formula (I) may be readily prepared by methods known in the art.

For example, the compound represented by formula (I) may be prepared according to Synthesis Examples described below.

The compound represented by formula (I) has a central structural unit of two arylene skeletons linked by a carbodiimide group with a perfluoropolyether group bonded to one end and a multifunctional (meth)acryloyl group bonded to the other end via urethane bonds, which can impart good initial contact angle and wear resistance while being soluble in solvents commonly used in clear coating agents such as methyl ethyl ketone (MEK), ethyl acetate (EA), and the like. In particular, the compound represented by formula (I) has a multifunctional (meth)acryloyl group, which provides excellent compatibility with (meth)acrylic-based clear coating agents. Furthermore, the compound represented by formula (I) can be cured together with other curable components contained in the (meth)acrylic-based clear coating agents to impart even better wear resistance. Thus, the compound represented by formula (I) can be usefully employed in (meth)acrylic-based clear coating agents as an additive to provide initial contact angle and wear resistance.

Thus, one embodiment of the present invention relates to a hard coating composition comprising the compound represented by formula (I).

The compound represented by formula (I) may be included in an amount of 2.5 to 10 wt %, preferably 3 to 5 wt %, based on 100 wt % of the total hard coating composition. If the compound represented by formula (I) is included in an amount of less than 2.5 wt % based on 100 wt % of the total hard coating composition, the initial contact angle and wear resistance may be deteriorated, and if it is included in an amount of more than 10 wt %, the properties of the film formed from the hard coating composition may be impaired.

The hard coating composition according to one embodiment of the present invention may further comprise a light-transmitting resin, a photoinitiator, and a solvent.

The light-transmitting resin is a light-curable resin, which may include, but is not limited to, a light-curable (meth)acrylate oligomer and/or monomer.

As the light-curable (meth)acrylate oligomer, epoxy (meth)acrylate, urethane (meth)acrylate, polyhedral oligomeric silsesquioxane (meth)acrylate, and the like can be used. In particular, urethane (meth)acrylate is preferred in terms of compatibility with the compound represented by formula (I) and improved wear resistance.

The urethane (meth)acrylate can be prepared by reacting a (meth)acrylate having a hydroxy group with a compound having an isocyanate group in the presence of a catalyst. Specific examples of the (meth)acrylate having an hydroxy group include 2-hydroxyethyl (meth)acrylate, 2-hydroxyisopropyl (meth)acrylate, 4-hydroxybutyl (meth)acrylate, caprolactone modified hydroxy (meth)acrylate, pentaerythritol tri(meth)acrylate, pentaerythritol tetra(meth)acrylate, dipentaerythritol penta(meth)acrylate, and the like. Further, specific examples of the compound having an isocyanate group include 1,4-diisocyanatobutane, 1,6-diisocyanatohexane, 1,8-diisocyanatooctane, 1,12-diisocyanatododecane, 1,5-diisocyanato-2-methylpentane, trimethyl-1,6-diisocyanatohexane, 1,3-bis(isocyanatomethyl)cyclohexane, trans-1,4-cyclohexenediisocyanate, 4,4′-methylene bis(cyclohexylisocyanate), isophorone diisocyanate, toluene-2,4-diisocyanate, toluene-2,6-diisocyanate, xylene-1,4-diisocyanate, tetramethylxylene-1,3-diisocyanate, 1-chloromethyl-2,4-diisocyanate, 4,4′-methylene bis(2,6-dimethylphenylisocyanate), 4,4′-oxybis(phenylisocyanate), trifunctional isocyanate derived from hexamethylene diisocyanate, trimethylolpropane adduct of toluene diisocyanate, and the like.

As the monomer, any one commonly used in the art can be used without limitation. Specifically, the monomer having an unsaturated group such as a (meth)acryloyl group, a vinyl group, a styryl group, an allyl group, etc. in the molecule as a light-curable functional group is preferred. In particular, the monomer having a (meth)acryloyl group is preferred in terms of compatibility with the compound represented by formula (I) and improved wear resistance.

By way of examples, the monomer having a (meth)acryloyl group may be one or more selected from the group consisting of neopentyl glycol (meth)acrylate, 1,6-hexanediol di(meth)acrylate, propylene glycol di(meth)acrylate, triethylene glycol di(meth)acrylate, dipropylene glycol di(meth)acrylate, polyethylene glycol di(meth)acrylate, polypropylene glycol di(meth)acrylate, trimethylolpropane tri(meth)acrylate, trimethylolethane tri(meth)acrylate, 1,2,4-cyclohexane tetra(meth)acrylate, pentaerythritol tri(meth)acrylate, pentaerythritol tetra(meth)acrylate, dipentaerythritol hexa(meth)acrylate, isooctyl(meth)acrylate, isodecyl (meth)acrylate, stearyl (meth)acrylate, tetrahydrofurfuryl (meth)acrylate, phenoxyethyl (meth)acrylate, and isoborneol (meth)acrylate.

Each of the above-exemplified light-curable (meth)acrylate oligomers and monomers may be used alone or in combination of two or more.

The light-transmitting resin may be included in an amount of 1 to 80 wt % based on 100 wt % of the total hard coating composition. When the content of the light-transmitting resin is less than 1 wt %, it is difficult to achieve sufficient hardness improvement, and when it exceeds 80 wt %, there is a problem of severe curling.

The photoinitiator is included for inducing photo-curing of the hard coating composition. The photoinitiator may include, for example, a photo-radical initiator capable of forming radicals by light irradiation.

Examples of the photoinitiator include a Type 1 initiator, which generates radicals by decomposition of molecules due to a difference in chemical structure or molecular binding energy, and a Type 2 initiator, which coexists with a tertiary amine to induce hydrogen abstraction.

For example, the Type 1 initiator may include acetophenones such as 4-phenoxydichloroacetophenone, 4-t-butyldichloroacetophenone, 4-t-butyltrichloroacetophenone, diethoxyacetophenone, 2-hydroxy-2-methyl-1-phenylpropan-1-one, 1-(4-isopropylphenyl)-2-hydroxy-2-methylpropan-1-one, 1-(4-dodecylphenyl)-2-hydroxy-2-methylpropan-1-one, 4-(2-hydroxyethoxy)-phenyl(2-hydroxy-2-propyl)ketone, and 1-hydroxycyclohexylphenylketone; benzoins such as benzoin, benzoin methyl ether, benzoin ethyl ether, and benzyl dimethyl ketal; phosphine oxides; titanocene compounds; and the like.

For example, the Type 2 initiator may include benzophenones such as benzophenone, benzoylbenzoic acid, benzoylbenzoic acid methyl ether, 4-phenylbenzophenone, hydroxybenzophenone, 4-benzole-4′-methyldiphenylsulfide, and 3,3′-methyl-4-methoxybenzophenone; thioxanthones such as thioxanthone, 2-chlorothioxanthone, 2-methylthioxanthone, 2,4-dimethylthioxanthone, isopropylthioxanthone; and the like.

The photoinitiators described above can be used alone or in a mixture of two or more. In addition, the above Type 1 and Type 2 initiators may be used alone or in combination thereof.

The photoinitiator may be included in an amount of about 0.1 to 10 wt %, preferably about 1 to 5 wt %, based on 100 wt % of the total hard coating composition. If the content of the photoinitiator is less than 0.1 wt %, sufficient curing may not occur, and the mechanical properties and adhesion of the hard coating film or hard coating layer may not be secured. If the content of the photoinitiator exceeds 10 wt %, poor adhesion, cracking, and curling due to cure shrinkage may result.

The solvent can be any solvent used in the art, without limitation. For example, alcohols (methanol, ethanol, isopropanol, butanol, methylcellosolve, ethylcellosolve, etc.), ketones (methylethyl ketone, methylbutyl ketone, methylisobutyl ketone, diethyl ketone, dipropyl ketone, cyclohexanone, etc.), acetates (ethyl acetate, propyl acetate, n-butyl acetate, t-butyl acetate, methylcellosolve acetate, ethylcellosolve acetate, propylene glycol monomethyl ether acetate, propylene glycol monoethyl ether acetate, propylene glycol monopropyl ether acetate, methoxybutyl acetate, methoxypentyl acetate, etc.), hexanes (hexane, heptane, octane, etc.), benzenes (benzene, toluene, xylene, etc.), ethers (diethylene glycol dimethyl ether, diethylene glycol diethyl ether, diethylene glycol dipropyl ether, diethylene glycol dibutyl ether, propylene glycol monomethyl ether, etc.) may be preferably used as the solvent. Each of the above-exemplified solvents can be used alone or in combination of two or more.

The solvent may be included in a residual amount to form 100 wt % of the total hard coating composition, for example in an amount of 10 to 95 wt % relative to 100 wt % of the total hard coating composition. If the content of the solvent is less than 10 wt %, the viscosity becomes high to make workability poor, and if it exceeds 95 wt %, the drying process becomes time-consuming and uneconomical.

In addition to the above components, the hard coating composition may further comprise components commonly used in the art, such as leveling agents, heat stabilizers, antioxidants, surfactants, lubricants, antifouling agents, and the like.

One embodiment of the present invention relates to a hard coating film formed using the hard coating composition described above. The hard coating film according to one embodiment of the present invention is characterized in that a coating layer comprising a cured product of the hard coating composition described above is formed on one side of a substrate.

The substrate can be used without particular limitation as long as it is a substrate used in the art. Specifically, a film having excellent transparency, mechanical strength, thermal stability, moisture barrier, isotropy, etc. can be used as the substrate. More specifically, the substrate may be a film made of a thermoplastic resin such as polyester-based resins such as polyethylene terephthalate, polyethylene isophthalate, polyethylene naphthalate, polybutylene terephthalate, and the like; cellulose-based resins such as diacetyl cellulose, triacetyl cellulose, and the like; polycarbonate-based resins; acrylic-based resins such as polymethyl (meth)acrylate, polyethyl (meth)acrylate, and the like; styrene-based resins such as polystyrene, acrylonitrile-styrene copolymers, and the like; polyolefin-based resins such as polyethylene, polypropylene, polyolefins with cyclo or norbornene structures, ethylene-propylene copolymers, and the like; vinyl chloride-based resins; amide-based resins such as nylon, aromatic polyamides, and the like; imide-based resins; sulfone-based resins; polyethersulfone-based resins; polyetheretherketone-based resins; polyphenylene sulfide-based resins; vinyl alcohol-based resins; vinylidene chloride-based resins; vinylbutyral-based resins; allylate-based resins; polyoxymethylene-based resins; epoxy-based resins; and the like. Also, films made of blends of the above thermoplastic resins can be used as the substrate. In addition, films composed of thermosetting resins or UV-curable resins such as (meth)acrylic, urethane, (meth)acrylurethane, epoxy, silicone, and the like, or ultra thin glass (UTG) can be utilized. According to one embodiment of the present invention, polyimide-based resins or polyester-based resins can be used, which are more resistant to repeated bending and more easily applicable to flexible image display devices.

The thickness of the substrate is not particularly limited, but may be from 8 to 1,000 μm, and particularly from 20 to 150 μm. If the thickness of the substrate is less than 8 μm, the strength of the film is reduced and the processability is deteriorated, and if it exceeds 1,000 μm, the transparency is reduced or the weight of the hard coating film is increased.

The hard coating film can be prepared by applying the hard coating composition described above on one surface of the substrate and curing it to form a coating layer.

The hard coating composition can be coated on the transparent substrate using any suitable method known in the art, such as a die coater, air knife, reverse roll, spray, blade, casting, gravure, micro gravure, spin coating, and the like.

After the hard coating composition is applied on the substrate, it is dried by evaporating volatiles at a temperature of 30 to 150° C. for 10 seconds to 1 hour, more specifically 30 seconds to 30 minutes, and then cured by irradiation with UV light. The irradiation amount of the UV light may be specifically about 0.01 to 10 J/cm², more specifically 0.1 to 2 J/cm².

At this time, the thickness of the coating layer formed may be specifically from 2 to 30 μm, more specifically from 3 to 20 μm. When the thickness of the coating layer is within the above range, an excellent hardness effect can be obtained.

One embodiment of the present invention relates to an image display device having the hard coating film described above. For example, the hard coating film of the present invention can be used as a window of an image display device, in particular a flexible display or a foldable display. Furthermore, the hard coating film of the present invention can be used by attaching it to a polarizing plate, a touch sensor, and the like.

The hard coating film according to one embodiment of the present invention can be used for reflective, transmissive and transflective LCDs, or LCDs of various driving types such as TN, STN, OCB, HAN, VA and IPS. The hard coating film according to one embodiment of the present invention can also be used for various image display devices such as plasma displays, field emission displays, organic EL displays, inorganic EL displays, electronic paper, etc.

One embodiment of the present invention relates to a composition for forming a low reflective layer comprising the compound represented by formula (I).

The compound represented by formula (I) may be included in an amount of 2.5 to 10 wt %, preferably 3 to 5 wt %, based on 100 wt % of the total composition for forming a low reflective layer. If the compound represented by formula (I) is included in an amount of less than 2.5 wt % based on 100 wt % of the composition for forming a low reflective layer, the initial contact angle and wear resistance may be deteriorated, and if it is included in an amount of more than 10 wt %, the properties of the film formed from the composition for forming a low reflective layer may be impaired.

The composition for forming a low-reflective layer according to one embodiment of the present invention may further comprise a light-transmitting resin, particulates having voids, a photoinitiator, and a solvent.

The particulates having voids make it possible to reduce the refractive index while maintaining the strength of the film formed from the composition for forming a low reflective layer.

By the particulates having voids are meant particulates having a gas-filled structure inside the particulates and/or a porous structure containing gas, such that the refractive index of the particulates decreases in inverse proportion to the share of gas in the particulates, relative to the original refractive index of the particulates.

The present invention also includes particulates that, by virtue of their morphology, structure, state of aggregation, and dispersion within the applied film, are capable of forming nanoporous structures within their interior and/or on at least a portion of their surface.

The particulates having voids are easy to manufacture and have high intrinsic hardness, so that the strength of the coating film is improved and at the same time the refractive index of the coating film can be adjusted in the range of 1.25 to 1.45.

The particulates having voids may specifically be a deterrent material for adsorbing various chemicals on the porous parts of the filling column and surface, porous particulates used for catalyst fixation, or dispersions or agglomerates of hollow particulates intended for incorporation into insulation materials or low dielectric materials. As more specific examples, those selected from commercially available hollow silica particles under the trade name ELECOM, a product of JGC Catalysts and Chemicals Ltd., aggregates of porous silica particulates under the trade names Nipsil or Nipgel, both products of Nippon Silica Industry Co., Ltd., and colloidal silica UP series (product name) manufactured by Nissan Chemical Industries, Ltd., which has a chain-like connected structure of silica particulates can be used.

The average particle diameter of the particulates having voids may be 5 nm to 300 nm, preferably 8 nm to 100 nm, and more preferably 40 nm to 80 nm. When the average particle diameter of the particulates having voids is within the above range, it becomes possible to impart excellent transparency to the coating film.

The particulates having voids may be included in an amount of 0.5 to 10 wt %, relative to 100 wt % of the total composition for forming a low reflective layer. When the particulates having voids are included in the above range, it is possible to adjust the refractive index of the coating film, and the anti-reflection effect is excellent.

The description of the types of the light-transmitting resin, the photoinitiator and the solvent is the same as described in the above hard coating composition, and therefore specific description is omitted.

The light-transmitting resin may be included in an amount of 1 to 80 wt %, relative to 100 wt % of the total composition for forming a low reflective layer. When the light-transmitting resin is included in the above range, the refractive index of the coating film is adjustable, and the anti-reflection effect is excellent.

The photoinitiator may be included in an amount of 0.1 to 10 wt %, preferably 1 to 5 wt %, relative to 100 wt % of the total composition for forming a low reflective layer. If the content of the photoinitiator is less than 0.1 wt %, the curing rate of the composition may be slow and undercuring may occur, resulting in poor mechanical properties, and if it exceeds 10 wt %, overcuring may occur, resulting in cracking of the coating film.

The solvent may be included in a residual amount to form 100 wt % of the total composition for forming a low reflective layer, for example, in an amount of 10 to 95 wt % relative to 100 wt % of the total composition for forming a low reflective layer. If the content of the solvent is less than 10 wt %, the viscosity becomes high to make workability poor, and if it exceeds 95 wt %, the drying process becomes time-consuming and uneconomical.

In addition to the above components, the composition for forming a low reflective layer may further comprise antioxidants, UV absorbers, light stabilizers, leveling agents, surfactants, antifouling agents, inorganic nanoparticles, and the like commonly used in the art, within the range that does not reduce the effects of the present invention.

One embodiment of the present invention relates to a coating film formed using the above-described composition for forming a low reflective layer.

The coating film can be formed by applying the composition for forming a low reflective layer on a substrate, followed by drying and UV curing.

The same substrates can be used as those used in the above hard coating composition.

The composition for forming a low reflective layer can be coated using any suitable method known in the art, such as a die coater, air knife, reverse roll, spray, blade, casting, gravure, micro gravure, spin coating, and the like.

After the composition for forming a low reflective layer is applied on the substrate, it is dried by evaporating volatiles at a temperature of 30 to 150° C. for 10 seconds to 1 hour, more specifically for 30 seconds to 30 minutes, and then cured by irradiating with UV light. The irradiation amount of the UV light may be specifically about 0.01 to 10 J/cm², more specifically 0.1 to 2 J/cm².

The thickness of the film may be 50 to 200 nm, preferably 50 to 100 nm.

One embodiment of the present invention relates to an image display device comprising the coating film described above. In this case, the coating film is used as a low reflective layer.

The description of the image display device is the same as that of the image display device with the hard coating film described above, and therefore specific description is omitted.

Hereinafter, the present invention will be described more specifically by means of examples, comparative examples, and experimental examples. These examples, comparative examples, and experimental examples are intended to illustrate the present invention only, and it is obvious to those skilled in the art that the scope of the present invention is not limited thereto.

### Synthesis Example 1: Synthesis of the Compound of Formula (I-1)

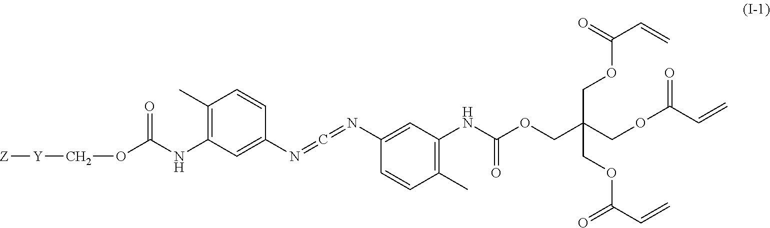

After adding 50.0 g of methyl ethyl ketone (MEK) to a 250 ml round bottom flask equipped with a reflux cooler, thermometer, and stirrer under an inert atmosphere, 8 g of 4,4′-ditosyl-carbodiimide-3,3′-diisocyanate was added, followed by adding 0.10 g of a 20% solution of dibutyl tin dilaurate (DBTDL) diluted in ethyl acetate and 0.02 g of 2,6-di-tert-butyl-4-methylphenol (BHT). Then, 4.0 g of perfluoropolyether alcohol represented by CF₃O(CF₂CF₂O)ₐ₁(CF₂O)ₐ₂CF₂CH₂OH (average MW=2358; a1/a2=1.14; (present in a ratio such that F is 1.29)) was added into the reactor and the temperature was raised to 55° C.

Subsequently, 40.0 g of pentaerythritol triacrylate was added and stirred for about 6 hours.

The degree of conversion was monitored by FT IR analysis monitoring the disappearance of the 2250 cm⁻¹ isocyanate absorption band and by F-NMR analysis observing the shift of the pre-terminal —CF₂— from −81.3 and −83.3 ppm (when connected to the —CH₂OH group) to −77.5 and −79.5 ppm (when connected to the moiety —CH₂OC(O)NH—). The solvent was then distilled under vacuum (10⁻¹ mbar and 60° C.) to obtain 48 g of the title compound.

### Synthesis Example 2: Synthesis of the Compound of Formula (I-2)

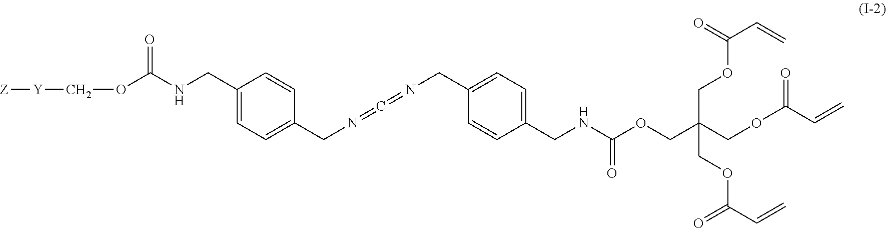

After adding 50.0 g of methyl ethyl ketone (MEK) to a 250 ml round bottom flask equipped with a reflux cooler, thermometer, and stirrer under an inert atmosphere, 9 g of bis(4-(isocyanatomethyl)benzyl)methanediimine was added, followed by adding 0.10 g of a 20% solution of dibutyl tin dilaurate (DBTDL) diluted in ethyl acetate and 0.02 g of 2,6-di-tert-butyl-4-methylphenol (BHT). Then, 4.0 g of perfluoropolyether alcohol represented by CF₃O(CF₂CF₂O)ₐ₁(CF₂O)ₐ₂CF₂CH₂OH (average MW=2358; a1/a2=1.14; (present in a ratio such that F is 1.29)) was added into the reactor and the temperature was raised to 55° C.

Subsequently, 40.0 g of pentaerythritol triacrylate was added and stirred for about 6 hours.

The degree of conversion was monitored by FT IR analysis monitoring the disappearance of the 2250 cm⁻¹ isocyanate absorption band and by F-NMR analysis observing the shift of the pre-terminal —CF₂— from −81.3 and −83.3 ppm (when connected to the —CH₂OH group) to −77.5 and −79.5 ppm (when connected to the moiety —CH₂OC(O)NH—). The solvent was then distilled under vacuum (10⁻¹ mbar and 60° C.) to obtain 51 g of the title compound.

### Synthesis Example 3: Synthesis of the Compound of Formula (I-3)

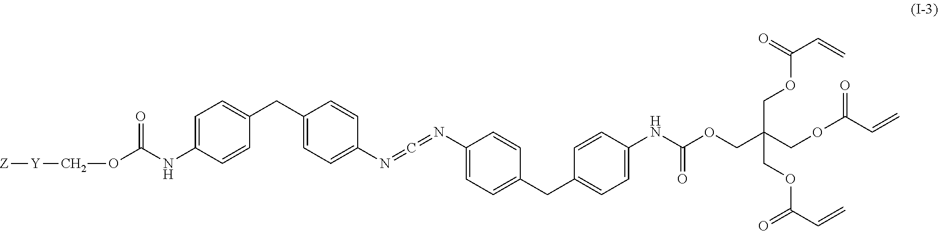

After adding 50.0 g of methyl ethyl ketone (MEK) to a 250 ml round bottom flask equipped with a reflux cooler, thermometer, and stirrer under an inert atmosphere, 10 g of bis(4-(isocyanatobenzyl)phenyl)methanediimide was added, followed by adding 0.10 g of a 20% solution of dibutyl tin dilaurate (DBTDL) diluted in ethyl acetate and 0.02 g of 2,6-di-tert-butyl-4-methylphenol (BHT). Then, 4.0 g of perfluoropolyether alcohol represented by CF₃O(CF₂CF₂O)ₐ₁(CF₂O)ₐ₂CF₂CH₂OH (average MW=2358; a1/a2=1.14; (present in a ratio such that F is 1.29)) was added into the reactor and the temperature was raised to 55° C.

Subsequently, 40.0 g of pentaerythritol triacrylate was added and stirred for about 6 hours.

The degree of conversion was monitored by FT IR analysis monitoring the disappearance of the 2250 cm⁻¹ isocyanate absorption band and by F-NMR analysis observing the shift of the pre-terminal —CF₂— from −81.3 and −83.3 ppm (when connected to the —CH₂OH group) to −77.5 and −79.5 ppm (when connected to the moiety —CH₂OC(O)NH—). The solvent was then distilled under vacuum (10-1 mbar and 60° C.) to obtain 52 g of the title compound.

### Synthesis Example 4: Synthesis of the Compound of Formula (I-4)

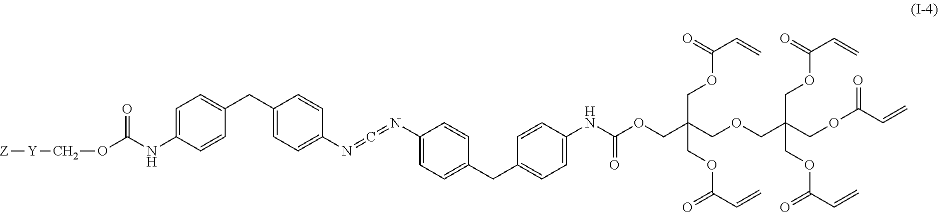

After adding 50.0 g of methyl ethyl ketone (MEK) to a 250 ml round bottom flask equipped with a reflux cooler, thermometer, and stirrer under an inert atmosphere, 10 g of bis(4-(isocyanatobenzyl)phenyl)methanediimide was added, followed by adding 0.10 g of a 20% solution of dibutyl tin dilaurate (DBTDL) diluted in ethyl acetate and 0.02 g of 2,6-di-tert-butyl-4-methylphenol (BHT). Then, 4.0 g of perfluoropolyether alcohol represented by CF₃O(CF₂CF₂O)ₐ₁(CF₂O)ₐ₂CF₂CH₂OH (average MW=2358; a1/a2=1.14; (present in a ratio such that F is 1.29)) was added into the reactor and the temperature was raised to 55° C.

Subsequently, 50.0 g of dipentaerythritol pentaacrylate was added and stirred for about 6 hours.

The degree of conversion was monitored by FT IR analysis monitoring the disappearance of the 2250 cm⁻¹ isocyanate absorption band and by F-NMR analysis observing the shift of the pre-terminal —CF₂— from −81.3 and −83.3 ppm (when connected to the —CH₂OH group) to −77.5 and −79.5 ppm (when connected to the moiety —CH₂OC(O)NH—). The solvent was then distilled under vacuum (10-1 mbar and 60° C.) to obtain 58 g of the title compound.

### Example 1

A hard coating composition was prepared by adding the compound of formula (I-1) obtained in Synthesis Example 1 in an amount of 5 wt % to a hard coating composition obtained by mixing 20 wt % of 3-functional acrylate (MIRAMER M340, Miwon), 20 wt % of 6-functional acrylate (UA-110H, Shinnakamura), 3.5 wt % of 1-hydroxycyclohexylphenylketone as a photoinitiator, and 51.5 wt % of methyl ethyl ketone (MEK) as a solvent.

The hard coating composition was coated on one surface of a polyester film (PET, 50 μm) to a thickness of 5 μm after curing, followed by drying the solvent and irradiating with a UV integrated light amount of 600 mJ/cm² under a nitrogen atmosphere to prepare a hard coating film.

### Example 2

A hard coating film was prepared in the same manner as in Example 1, except that the compound of formula (I-2) obtained in Synthesis Example 2 was used instead of the compound of formula (I-1).

### Example 3

A hard coating film was prepared in the same manner as in Example 1, except that the compound of formula (I-3) obtained in Synthesis Example 3 was used instead of the compound of formula (I-1).

### Example 4

A hard coating film was prepared in the same manner as in Example 1, except that the compound of formula (I-4) obtained in Synthesis Example 4 was used instead of the compound of formula (I-1).

### Example 5

A hard coating film was prepared in the same manner as in Example 3, except that the compound of formula (I-3) was added in an amount of 3 wt % instead of 5 wt %, and methyl ethyl ketone (MEK) was added in an amount of 53.5 wt % as a solvent.

### Comparative Example 1

A hard coating film was prepared in the same manner as in Example 1, except that a hard coating composition comprising a solvent in an amount of 56.5 wt % was used, without adding the compound of formula (I-1).

### Comparative Example 2

A hard coating film was prepared in the same manner as in Example 1, except that KY-1203 (Shin-Etsu Chemical Co., Ltd., antifouling agent) was used instead of the compound of formula (I-1).

### Example 6

A low refractive index composition was prepared by mixing 2 wt % of dipentaerythritol hexaacrylate, 6 wt % of hollow silica particulates (JGC Catalysts and Chemicals Ltd., THRULYA4320, 20 wt % solids), 0.5 wt % of a photoinitiator (BASF, Irgacure 184), 86.2 wt % of propylene glycol monomethyl ether as a solvent, 0.3 wt % of a leveling agent (BYK company, BYKUV3570), and 5 wt % of the compound of formula (I-1) obtained in Synthesis Example 1.

The low refractive index composition was coated on the hard coating layer of the hard coating film prepared in Comparative Example 1 so that the thickness was 80 nm after curing, followed by drying the solvent and irradiating with a UV integrated light amount of 600 mJ/cm² under a nitrogen atmosphere to prepare a low reflective film.

### Example 7

A low reflective film was prepared in the same manner as in Example 6, except that the compound of formula (I-2) obtained in Synthesis Example 2 was used instead of the compound of formula (I-1).

### Example 8

A low reflective film was prepared in the same manner as in Example 6, except that the compound of formula (I-3) obtained in Synthesis Example 3 was used instead of the compound of formula (I-1).

### Example 9

A low reflective film was prepared in the same manner as in Example 6, except that the compound of formula (I-4) obtained in Synthesis Example 4 was used instead of the compound of formula (I-1).

### Example 10

A low reflective film was prepared in the same manner as in Example 6, except that the compound of formula (I-1) was added in an amount of 3 wt %, and the solvent was added in an amount of 88.2 wt %.

### Comparative Example 3

A low reflective film was prepared in the same manner as in Example 6, except that the compound of formula (I-1) was not added, and a solvent was added in an amount of 91.2 wt %.

### Experimental Example 1

The properties of the hard coating films and the low reflective films prepared in the above examples and comparative examples were measured by the following methods, and the results are shown in Table 1 below.

### (1) Contact Angle

The water contact angle was measured using a contact angle meter DSA100 (KRUSS GmbH). The liquid drop volume was 2 [symbol] at room temperature.

### (2) Scratch Test

The substrate film was bonded to a glass with the coating side facing upward using a transparent adhesive. Then, a steel wool (#0000) was used to rub reciprocally 10 times with a load of 500 g/cm², and the number of scratches was checked by transmitting and reflecting the measured area under a tri-wavelength lamp.

### (3) Steel Wool Test

The substrate film was bonded to a glass with the coating side facing upward using a transparent adhesive. Then, a steel wool (#0000) was used to rub reciprocally with a load of 500 g/cm², and the number of times the contact angle was 100° or more was counted.

### (4) Eraser Test

The eraser test was conducted using the wear measurement equipment of Daesung Precision. Using an eraser (Minoan safety apparatus) with a hardness of 88 and a diameter of 6 mm, the coating surface was reciprocally rubbed 2,000 times with a load of 1 kg, and the number of times the contact angle was 100° or more was counted.

TABLE 1

| | Initial | Wear resistance assessment | | |
|---|---|---|---|---|
| | contact | Scratch | Steel wool | Eraser |
| | angle | test | test | test |
| Example 1 | 112.8 | 2 | 20,000 | 8,000 |
| Example 2 | 113.1 | 3 | 18,000 | 8,000 |
| Example 3 | 113.6 | 2 | 20,000 | 10,000 |
| Example 4 | 113.2 | 2 | 20,000 | 10,000 |
| Example 5 | 112.4 | 2 | 18,000 | 8,000 |
| Example 6 | 112.9 | 2 | 11,000 | 7,000 |
| Example 7 | 113.0 | 2 | 9,000 | 6,000 |
| Example 8 | 113.5 | 3 | 10,000 | 7,000 |
| Example 9 | 113.2 | 2 | 10,000 | 5,000 |
| Example 10 | 111.9 | 3 | 8,000 | 6,000 |
| Comparative | 57.2 | 5 | No test | No test |
| Example 1 | | | | |
| Comparative | 110.2 | 4 | 6,000 | 2,000 |
| Example 2 | | | | |
| Comparative | 79.4 | 5 | No test | No test |
| Example 3 | | | | |

From Table 1 above, it can be seen that the hard coating films of Examples 1 to 5 formed from the hard coating composition comprising the compound of formula (I) have a higher initial contact angle as well as better wear resistance, as compared to the hard coating film of Comparative Example 2 formed from the hard coating composition comprising a conventional antifouling agent instead of the compound of formula (I).

It can also be seen that the low reflective films of Examples 6 to 10 formed from the composition for forming a low reflective layer comprising the compound of formula (I) not only have a high initial contact angle, but also have excellent wear resistance.

On the other hand, the hard coating film of Comparative Example 1 formed from a hard coating composition that does not include the compound of formula (I) and the low reflective film of Comparative Example 3 formed from a composition for forming a low reflective layer that does not include the compound of formula (I) were found to have a significantly poor initial contact angle, so the steel wool test and the eraser test were not conducted.

Although particular embodiments of the present invention have been shown and described, it will be understood by those skilled in the art that it is not intended to limit the present invention to the preferred embodiments, and it will be obvious to those skilled in the art that various changes and modifications may be made without departing from the spirit and scope of the invention.

The scope of the present invention, therefore, is to be defined by the appended claims and equivalents thereof.

1. A compound represented by the following formula (I): 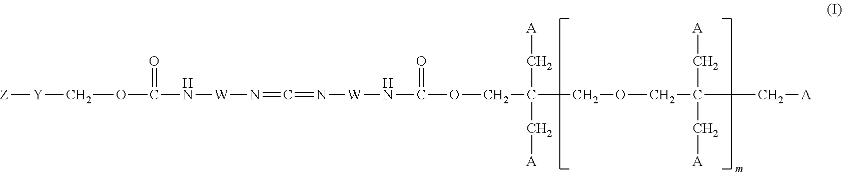
wherein,
Z is C₁-C₁₆ alkyl unsubstituted or substituted with fluorine;
Y represents —(OC₄F₈)ₐ—(OC₃F₆)_{b}—(OC₂F₄)_{c}—(OCF₂)_{d}—, wherein a, b, c, and d are each independently an integer from 0 to 200, and the sum of a, b, c, and d is 5 or more;
W represents 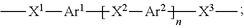
Ar¹ and Ar² are each independently arylene unsubstituted or substituted with C₁-C₄ alkyl, C₁-C₄ alkoxy, hydroxyl, or halogen;
X¹ to X³ are each independently absent, or C₁-C₄ alkylene unsubstituted or substituted with oxygen, sulfur, sulfonyl, or C₁-C₄ haloalkyl;
A is (meth)acryloyloxy; and
n and m are each independently 0 or 1.

2. The compound according to claim 1, wherein Ar¹ and Ar² are each independently phenylene unsubstituted or substituted with C₁-C₄ alkyl.

3. The compound according to claim 1, wherein X¹ to X³ are each independently be absent, or C₁-C₄ alkylene unsubstituted or substituted with C₁-C₄ haloalkyl.

4. The compound according to claim 1, wherein the compound is represented by any one of the following formulas (I-1) to (I-4): 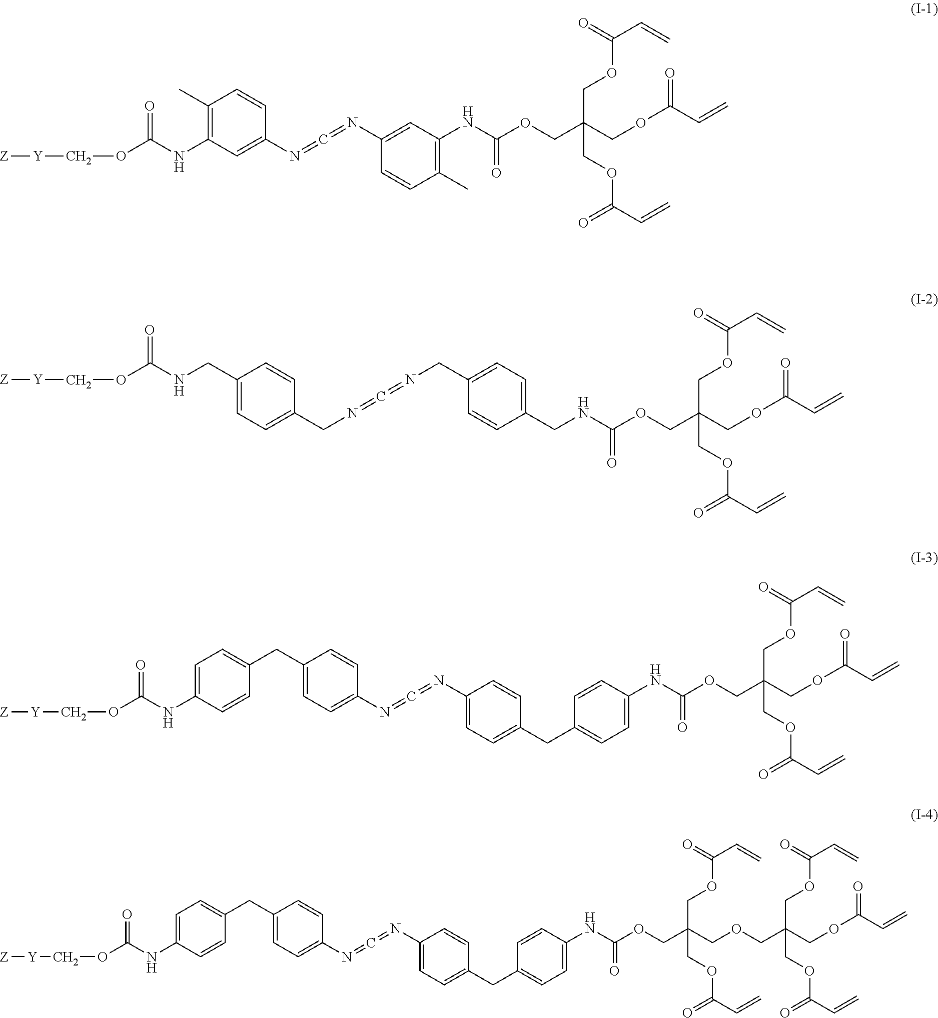
wherein,
Z is C₁-C₁₆ alkyl unsubstituted or substituted with fluorine; and
Y represents —(OC₄F₈)ₐ—(OC₃F₆)_{b}—(OC₂F₄)_{c}—(OCF₂)_{d}—, wherein a, b, c, and d are each independently an integer from 0 to 200, and the sum of a, b, c, and d is 5 or more.

5. A hard coating composition comprising the compound according to claim 1.

6. The hard coating composition according to claim 5, wherein the compound according to claim 1 is included in an amount of 2.5 to 10 wt % based on 100 wt % of the total hard coating composition.

7. The hard coating composition according to claim 5, further comprising a light-transmitting resin, a photoinitiator, and a solvent.

8. A hard coating film formed using the hard coating composition according to claim 5.

9. An image display device having the hard coating film according to claim 8.

10. A window of a flexible display having the hard coating film according to claim 8.

11. A polarizing plate having the hard coating film according to claim 8.

12. A touch sensor having the hard coating film according to claim 8.

13. A composition for forming a low reflective layer comprising the compound according to claim 1.

14. The composition for forming a low reflective layer according to claim 13, wherein the compound according to claim 1 is included in an amount of 2.5 to 10 wt % based on 100 wt % of the total composition for forming a low reflective layer.

15. The composition for forming a low reflective layer according to claim 13, further comprising a light-transmitting resin, particulates having voids, a photoinitiator, and a solvent.

16. A coating film formed using the composition for forming a low reflective layer according to claim 13.

17. An image display device having the coating film according to claim 16.
